(19) United States
(12) Patent Application Publication
(10) Pub. No.: US 2008/0281305 A1
(43) Pub. Date: Nov. 13, 2008

(54) METHOD AND APPARATUS FOR RELIEVING ANGINA SYMPTOMS USING LIGHT

(75) Inventors: Tamara Colette Baynham, Blaine, MN (US); Darrell O. Wagner, Isanti, MN (US); Haris J. Sih, Minneapolis, MN (US)

Correspondence Address: SCHWEGMAN, LUNDBERG & WOESSNER, P.A., P.O. BOX 2938, MINNEAPOLIS, MN, 55402, US

(73) Assignee: Cardiac Pacemakers, Inc., St paul, MN (US)

(21) Appl. No.: 11/746,829

(22) Filed: May 10, 2007

(51) Int. Cl. A61B 18/18; A61F 2/06; A61F 7/12; A61N 5/06

(52) U.S. Cl. 606/10; 606/2; 607/113; 607/88; 623/1.15

(57) ABSTRACT

An implantable medical device includes a light emitting circuit incorporated into an intravascular stent. The light emitting circuit emits a light to an ischemic region. The light has characteristics suitable for reliving the angina symptoms associated with ischemia.

### TECHNICAL FIELD

This document relates generally to implantable medical devices and particularly a system for relieving angina symptoms using an implantable device that emits light to an ischemic region.

### BACKGROUND

The heart is the center of a person's circulatory system. It includes an electro-mechanical system performing two major pumping functions. The left portions of the heart draw oxygenated blood from the lungs and pump it to the organs of the body to provide the organs with their metabolic needs for oxygen. The right portions of the heart draw deoxygenated blood from the body organs and pump it to the lungs where the blood gets oxygenated. These pumping functions are resulted from contractions of the myocardium. In a normal heart, the sinoatrial node, the heart's natural pacemaker, generates electrical impulses that propagate through an electrical conduction system to various regions of the heart to excite the myocardial tissues of these regions. Coordinated delays in the propagations of the electrical impulses in a normal electrical conduction system cause the various portions of the heart to contract in synchrony to result in efficient pumping functions.

Cardiac ischemia is a condition in which the myocardium is deprived of adequate oxygen and metabolite removal due to reduced or interrupted blood supply caused by constriction of a blood vessel such as a coronary artery. In a patient having cardiac ischemia due to obstruction to blood flow in a coronary artery, angina (angina pectoris, or cardiac pain) is likely to develop when the blood flow fails to meet the metabolic need of the heart. Angina is also an indication that the cardiac ischemia may develop into myocardial infarction (MI). MI is the necrosis of portions of the myocardial tissue. The necrotic tissue, known as infarcted tissue, loses the contractile properties of the normal, healthy myocardial tissue. Consequently, the overall contractility of the myocardium is weakened, resulting in an impaired hemodynamic performance. Following an MI, cardiac remodeling starts with expansion of the region of infarcted tissue and progresses to a chronic, global expansion in the size and change in the shape of the entire left ventricle. The consequences include a further impaired hemodynamic performance and a significantly increased risk of developing heart failure, as well as a risk of suffering recurrent MI.

Therefore, there is a need to treat cardiac ischemia, including the associated angina symptoms.

### SUMMARY

An implantable medical device includes a light emitting circuit incorporated into an intravascular stent. The light emitting circuit emits a light to an ischemic region to relive angina symptoms associated with ischemia.

In one embodiment, the light emitting circuit includes a light source, a power supply circuit, and an implant controller. The light source emits a light having characteristics suitable for reliving the angina symptoms. The power supply circuit produces a power supply signal suitable for powering the light source. The implant control circuit controls the emission of the light from the light source during a light emission duration.

In one embodiment, a method for emitting a light to an ischemic region to relieve angina symptoms is provided. A light emission duration is started and timed. A light source is incorporated into an intravascular stent configured to be placed in the ischemic region, and is powered using a power source incorporated into the intravascular stent. The light is emitted from the light source during the light emission duration. The light has characteristics suitable for reliving the angina symptoms.

This Summary is an overview of some of the teachings of the present application and not intended to be an exclusive or exhaustive treatment of the present subject matter. Further details about the present subject matter are found in the detailed description and appended claims. The scope of the present invention is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 is an illustration of an embodiment of a system for relieving angina symptoms and portions of the environment in which the system operates.

FIG. 2 is an illustration of an embodiment of an implantable medical device and an intravascular stent delivery catheter assembly, where the implantable medical device includes an intravascular stent mounted on an expandable element of the intravascular stent delivery catheter assembly.

FIG. 3 is an illustration of an embodiment of the implantable medical device with the expandable element in an expanded state.

FIG. 4 is an illustration of an embodiment of the implantable medical device after withdrawal of the intravascular stent delivery catheter assembly.

FIG. 5 is an illustration of an embodiment of an implantable medical device including a light emitting circuit incorporated into an intravascular stent.

FIG. 6 is an illustration of another embodiment of an implantable medical device including a light emitting circuit incorporated into an intravascular stent.

FIG. 7 is an illustration of an embodiment of a polymeric light emitting diode (PLED) incorporated into an intravascular stent.

FIG. 8 is a block diagram illustrating an embodiment of a light emitting circuit for relieving angina symptoms.

FIG. 9 is a block diagram illustrating an embodiment of an implantable medical device for relieving angina symptoms.

FIG. 10 is a block diagram illustrating an embodiment of a light emitting circuit of the implantable medical device of FIG. 9.

FIG. 11 is a block diagram illustrating an embodiment of an external system.

FIG. 12 is a block diagram illustrating an embodiment of an external system.

FIG. 13 is a block diagram illustrating an embodiment of an implant control circuit of the implantable medical device of FIG. 9.

FIG. 14 is a block diagram illustrating an embodiment of an implant control circuit of the implantable medical device of FIG. 9 and an external control circuit of the external system of FIG. 11.

FIG. 15 is a block diagram illustrating an embodiment of an external control circuit of the external system of FIG. 11.

FIG. 16 is a flow chart illustrating a method for relieving angina symptoms using a light therapy.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses a system including an implantable medical device that delivers a light therapy to reduce angina symptoms resulting from ischemia. Examples of known therapies for treating angina symptoms caused by cardiac ischemia include spinal cord stimulation and enhanced external counterpulsation (EECP). Spinal cord stimulation suppresses the cardiac pain, but does not treat cardiac ischemia. EECP is known to treat cardiac ischemia with its angiogenetic effect, but requires a lengthy therapy (five hours per day for sevens days, for example). The present system uses a light emitting circuit incorporated into an intravascular stent. After the intravascular stent is placed in or near an ischemic region, the light emitting circuit provides for controlled emission of a light having characteristics suitable for reliving the angina symptoms. This therapy provides for pain relief and angiogenesis using relatively short periods of delivery. The angiogenetic effect has the potential of eventually eliminating the cause of the angina symptoms.

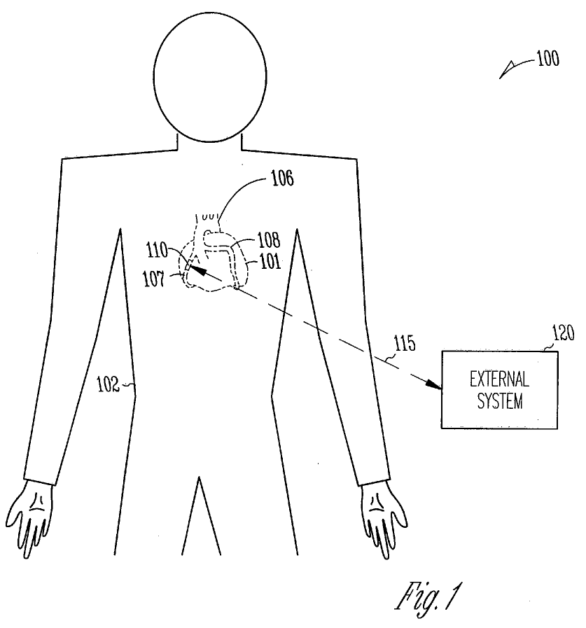

FIG. 1 is an illustration of an embodiment of a system 100 for relieving angina symptoms and portions of the environment in which system 100 operates. System 100 includes an implantable medical device 110, an external system 120, and a telemetry link 115. In the illustrated embodiment, implantable medical device 110 delivers a light therapy using an intravascular stent-based device that emits a light to treat angina pectoris resulting from cardiac ischemia. FIG. 1 illustrates a patient's body 102 having a heart 101 connected to an aorta 106. A right coronary artery 107 and a left coronary artery 108, which branch from aorta 106, supply heart 101 with oxygenated blood for its metabolic needs. Implantable medical device 110 is used to deliver the light therapy in response to a cardiac ischemia caused by, for example, narrowing or blockage of one of right coronary artery 107 and left coronary artery 108. In the illustrated embodiment, implantable medical device is inserted into right coronary artery 107 to open up that artery and treats the angina symptoms associated with the cardiac ischemia by delivering the light therapy. In another embodiment, implantable medical device is inserted into left coronary artery 108 to deliver the light therapy. The location where implantable medical device 110 is implanted depends on where the ischemic region is. In the illustrated embodiment, implantable medical device 110 is placed in a portion of an artery where plaque has built up. In one embodiment, one or more medical imaging techniques, such as positron-emission tomography (PET), are applied to identify the ischemic region or other area to which the light therapy is to be delivered and to assist the placement of implantable medical device 110.

External system 120 allows control of the delivery of the light therapy from implantable medical device 110. Telemetry link 115 provides for power and/or data transmission from external system 120 to implantable medical device 110. In one embodiment, telemetry link 115 uses a carrier signal to transmit power to implantable medical device 110 for the operation of the light emitting circuit, and the carrier signal is modulated for the date transmission. Examples of the carrier signal include magnetic signal and ultrasonic signal. In another embodiment, telemetry link 115 uses a carrier signal for power transmission and another carrier signal modulated for data transmission. For example, telemetry link 115 uses a magnetic or ultrasonic signal for power transmission and an electromagnetic signal modulated for data transmission. In another embodiment, telemetry link 115 also provides for data transmission from implantable medical device 110 to external system 120, such as for transmission of data representative of operation status of implantable medical device 110 and/or one or more signals sensed by implantable medical device 110.

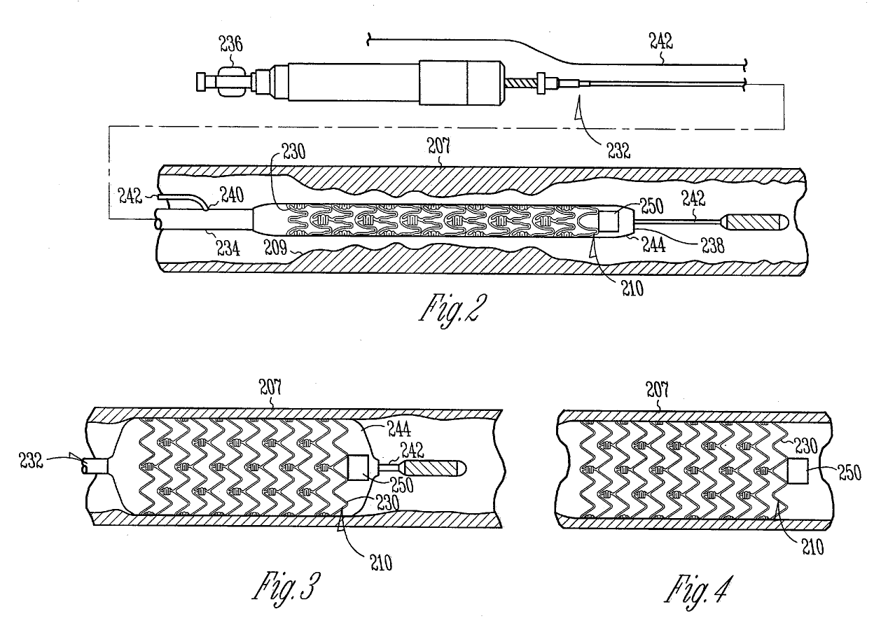

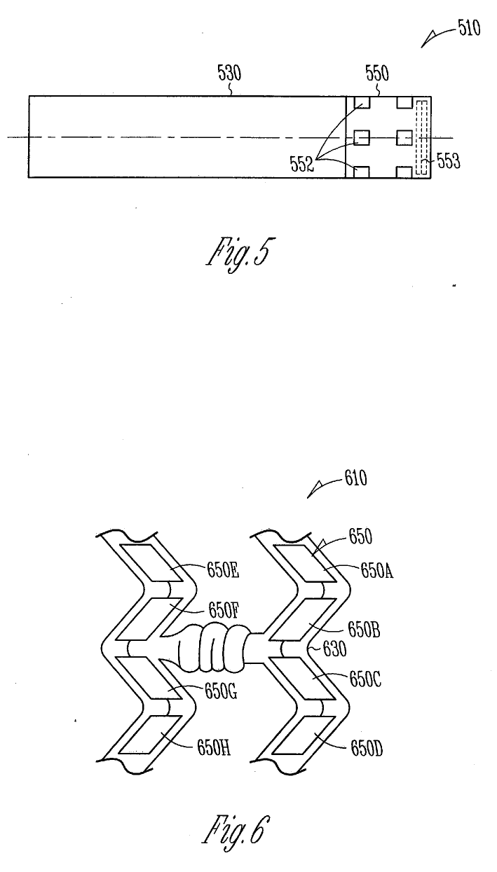

FIGS. 2-4 are illustrations of an embodiment of an implantable medical device 210 and an intravascular stent delivery intravascular stent delivery catheter assembly 232. Implantable medical device 210 represents an embodiment of implantable medical device 110. FIG. 2 illustrates implantable medical device 210 including an intravascular stent 230 and a light emitting circuit 250 incorporated into intravascular stent 230. Intravascular stent 230 as shown in FIG. 2 is mounted on an expandable element 244 of intravascular stent delivery catheter assembly 232, which is inserted into an artery 207. Examples of artery 207 include right coronary artery 107 and left coronary artery 108. FIG. 3 illustrates implantable medical device 210 with expandable element 244 in an expanded state. FIG. 4 illustrates implantable medical device 210 after the withdrawal of intravascular stent delivery catheter assembly 232 from artery 207.

Intravascular stent delivery catheter assembly 232 includes a catheter shaft 234, which has a proximal end 236 and a distal end 238. Intravascular stent delivery catheter assembly 232 is configured to advance through the patient's vascular system over a guide wire 242. Intravascular stent delivery catheter assembly 232 as illustrated in FIG. 2 is of a rapid exchange type which includes a port 240 where guide wire 242 exits catheter shaft 234. The distal end of guide wire 242 exits distal end 238 so that a section of catheter shaft 234 (between port 240 and distal end 238) advances over guide wire 242. Intravascular stent 230 is mounted on expandable element 244 and crimped thereon such that intravascular stent 230 and expandable element 244 present a low profile diameter for delivery through the vascular system.

Artery 207 as shown in FIG. 2 has plaque 209 that has been treated by a plaque-removal procedure such as angioplasty. Intravascular stent 230 is applied to prevent plaque 209 from narrowing artery 207 again to an extent of causing cardiac ischemia. In one embodiment, to position intravascular stent 230 during the implantation of implantable medical device 210, guide wire 242 is advanced through the patient's vascular system until its distal end is advanced past plaque 209 in artery 207. Intravascular stent delivery catheter assembly 232 is then advanced over guide wire 242 until intravascular stent 230 is positioned in a portion of artery 207 where plaque 209 is present. Expandable element 244, such as a balloon is expanded to expand intravascular stent 230 until intravascular stent 230 presses against the interior wall of artery 207, as illustrated in FIG. 3. Expandable element 244 is then contracted, and intravascular stent delivery catheter assembly 232 is withdrawn from the patient's vascular system. As illustrated in FIG. 4, intravascular stent 230 remains in the artery 207 to maintain its patency after the withdrawal of intravascular stent delivery catheter assembly 232.

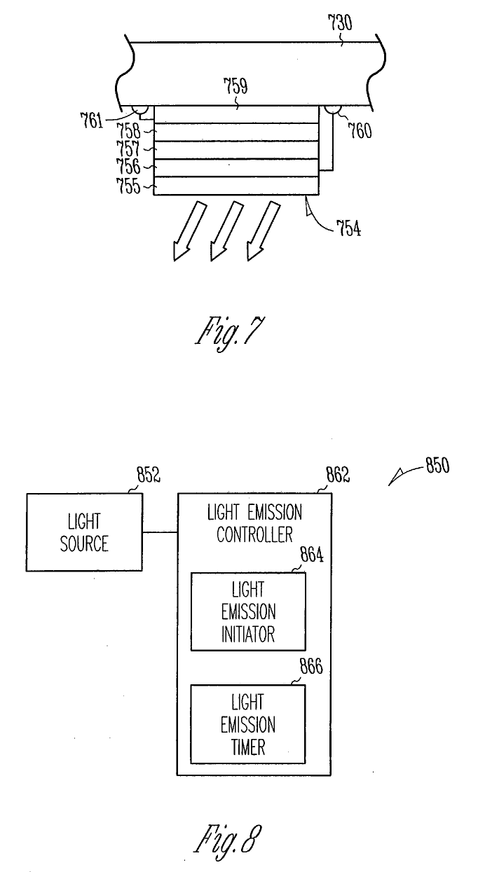

Light emitting circuit 250 is shown in FIG. 2, for illustration purposes only, to indicate that it is integral part of implantable medical device 210 and coupled to intravascular stent 230. Examples showing how a light emitting circuit incorporated into an intravascular stent is incorporated into an intravascular stent are discussed below, with reference to FIGS. 5-7.

FIG. 5 is an illustration of an embodiment of an implantable medical device 510. Implantable medical device 510 represents an embodiment of implantable medical device 110 and includes an intravascular stent 530 and a light emitting circuit 550 incorporated into intravascular stent 530. An example of intravascular stent 530 includes intravascular stent 230. In the illustrated embodiment, light emitting circuit 550 includes a plurality of light sources 552, such as light emitting diodes (LEDs), and a coil 553 to receive the power and/or data transmission signal transmitted from external system 120 via an inductive couple of telemetry link 115. In the illustrated embodiment, light emitting circuit 550 is constructed on a tubular substrate coupled to an end portion of intravascular stent 530. In other embodiments, light emitting circuit 550 is incorporated into intravascular stent 530 in any suitable manner that does not adversely affect blood flow through intravascular stent 530.

FIG. 6 is an illustration of an embodiment of portions of an implantable medical device 610. Implantable medical device 610 represents another embodiment of implantable medical device 110 and includes an intravascular stent 630 and a light emitting circuit 650 incorporated into intravascular stent 630. An example of intravascular stent 630 includes intravascular stent 230. FIG. 6 shows portions of a mesh of intravascular stent 630 and portions of light emitting circuit 650 (represented by 650A-H) each constructed on a substrate etched onto a portion of the mesh. In various embodiments, portions of light emitting circuit 650A-H are constructed on either one or both surfaces of intravascular stent 630, which include the external surface that contacts the wall of the artery and the opposite luminal side.

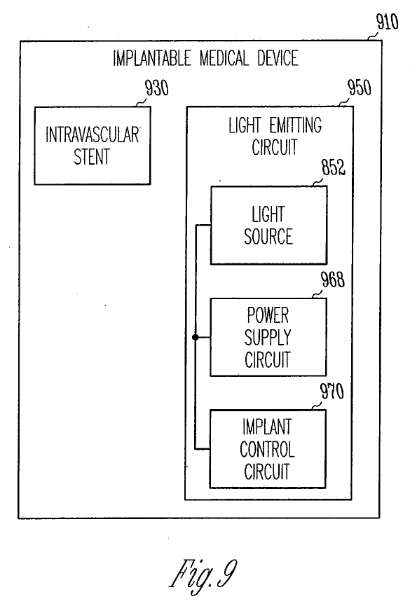

FIG. 7 is an illustration of an embodiment of a polymeric light emitting diode (PLED) 754 incorporated into an intravascular stent 730. An example of intravascular stent 730 includes intravascular stent 230. In one embodiment, PLED 754 is used as a light source of light emitting circuit 650. In another embodiment, PLED 754 is used as one of light sources 552.

In the illustrated embodiment, PLED 754 includes a transparent substrate 755 (such as a glass layer), a transparent anode 756 (such as an indium tin oxide layer) on transparent substrate 755, a hole transporting layer 757 on transparent anode 756, a light emitting polymer 758 on hole transporting layer 757, and a cathode 759 on light emitting polymer 758. PLED 754 emits a light for reliving the angina symptoms when a voltage at a specified level is applied using a conductor 760 connected to transparent anode 756 and another conductor 761 connected to cathode 759. In one embodiment, PLED 754 is mounted on a portion of intravascular stent 730, with cathode 759 in contact with a portion of the mesh of intravascular stent 730.

FIG. 8 is a block diagram illustrating an embodiment of a light emitting circuit 850 for relieving angina symptoms. Light emitting circuit 850 includes a light source 852 and a light emission controller 862. Examples of light source 852 include light source 552 and PLED 754. Light emitting circuits 250, 550, and 650 each include portions of light emitting circuit 850.

Light source 852 is configured for placement in a patient having angina symptoms to deliver the light therapy to an ischemic region in the patient's body. The light has characteristics suitable for reliving angina symptoms. In one embodiment, light source 852 is configured to emit a light having a wavelength between 600 nanometers and 1,000 nanometers, with approximately 800 nanometers being a specific example. Such a light has been experimentally shown to result in expression of vascular endothelial growth factors (VEGF) that may eventually trigger angiogenesis in the ischemic area. In one embodiment, light source 852 emits such a light in a gene therapy to enhance expression of light-sensitive promoters.

Light emission controller 862 controls the emission of the light from light source 852 and includes a light emission initiator 864 and a light emission timer 866. Light emission initiator 864 produces a light initiation signal to start a light emission duration during which light source 852 emits the light. In one embodiment, light emission initiator 864 receives a light emission command and produces the light initiation signal in response to the light emission command. In another embodiment, light emission initiator 864 is programmed to produce the light initiation signal according to a specified schedule, such as on a periodic basis using a programmed period. In one embodiment, the period is programmable between 1 minute and 72 hours. In one embodiment, light emission initiator 864 is programmed to produce the light initiation signal when the patient is most likely inactive, such as during normal sleeping time.

Light emission timer 866 produces a light emission signal in response to the light initiation signal. The light emission signal is present during the light emission duration. In other words, light source 852 emits the light while the light emission signal is present. In one embodiment, the light emission duration is programmable between 10 seconds and 60 minutes.

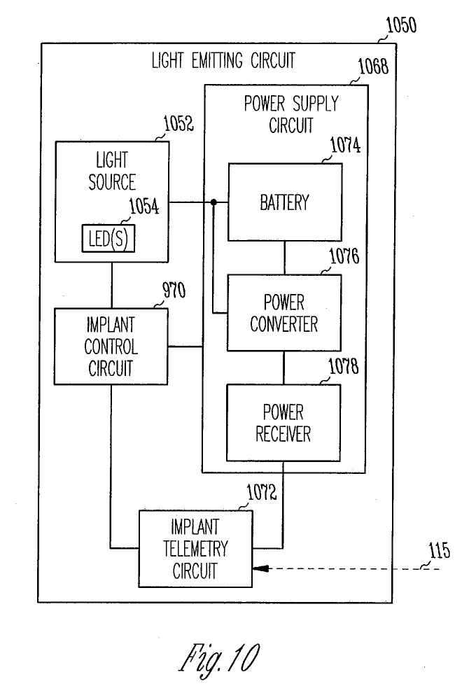

FIG. 9 is a block diagram illustrating an embodiment of an implantable medical device 910, which represents an embodiment of implantable medical device 110, 210, 510, or 610. Implantable medical device 910 includes an intravascular stent 930 and a light emitting circuit 950. An example of intravascular stent 930 includes intravascular stent 230. Light emitting circuit 950 is incorporated into intravascular stent 930 and includes light source 852, a power supply circuit 968, and an implant control circuit 970. Power supply circuit 968 produces a power supply signal suitable for powering light source 852 and implant control circuit 970, such as a DC signal having a specified voltage. Implant control circuit 970 controls the emission of the light from light source 852 during the light emission duration.

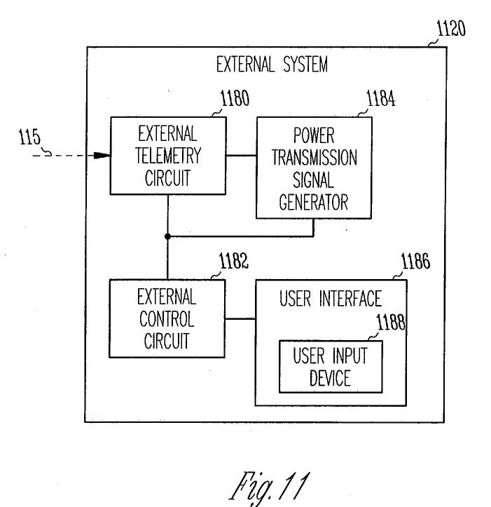

FIG. 10 is a block diagram illustrating an embodiment of a light emitting circuit 1050, which represents a specific embodiment of light emitting circuit 950. Light emitting circuit 1050 includes a light source 1052, a power supply circuit 1068, implant control circuit 970, and an implant telemetry circuit 1072.

Light source 1052 represents a specific embodiment of light source 852 and includes one or more LEDs 1054. In one embodiment, LED(s) 1054 include LED(s) in die form suitable for mounting on an intravascular stent. In another embodiment, LED(s) 1054 include PLED(s).

Power supply circuit 1068 represents a specific embodiment of power supply circuit 968. In the illustrated embodiment, power supply circuit 1068 includes a battery 1074, a power converter 1076, and a power receiver 1078. Power receiver 1078 receives the power transmission signal via telemetry link 115. Power converter 1076 produces the power supply signal suitable for powering light source 1052 and implant control circuit 970 using the power transmission signal. In a specific embodiment, power converter includes an AC-to-DC converter and a voltage regulator to convert the power transmission signal (such as a sinusoidal signal or a square-wave AC signal) to a DC signal having a specified voltage. In a specific embodiment, battery 1074 includes a rechargeable battery that is rechargeable using the power supply signal. In another embodiment, power supply circuit 1068 includes only battery 1074, for example, when implantable medical device 910 is intended for short-term use. In another embodiment, power supply circuit 1068 includes only power converter 1076 and power receiver 1078. Light source 1052 emits a light when the power transmission signal is being received via telemetry link 115.

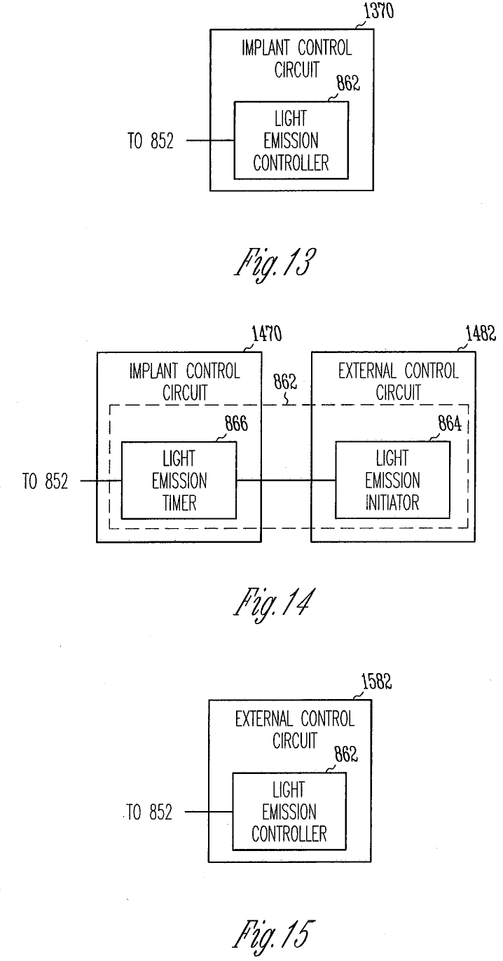

Implant telemetry circuit 1072 receives the power transmission signal. In one embodiment, implantable telemetry circuit 1072 also receives a data transmission signal including, for example, the light emission command that triggers the light initiation signal, the light initiation signal, or the light emission signal, depending on which portions of light emission controller 862 is included in implant control circuit 870, as further discussed below with reference to FIGS. 13-15. In one embodiment, the power transmission signal is used as the carrier signal for the data transmission. That is, the data transmission signal is the power transmission signal modulated by data representing the light emission command, the light initiation signal, or the light emission signal.

FIG. 11 is a block diagram illustrating an embodiment of an external system 1120, which represents an embodiment of external system 120. External system 1120 includes an external telemetry circuit 1180, an external control circuit 1182, a power transmission signal generator 1184, and a user interface 1186. External telemetry circuit 1180 transmits the power transmission signal to implantable medical device 910 via telemetry 115. Power transmission signal generator 1184 generates the power transmission signal. In one embodiment, external telemetry circuit 1180 also transmits the data transmission signal to implantable medical device 910 via telemetry 115, such as by modulating the power transmission signal. External control circuit 1182 controls the operation of external system 1120. In one embodiment, external control circuit 1182 includes portions of light emission controller 862, as further discussed below with reference to FIGS. 13-15. User interface 1186 allows a user, such as the patient or a physician or other caregiver to control the delivery of the light therapy from implantable medical device 910. In the illustrated embodiment, user interface 1186 includes a user input 1188 to receive the light emission command.

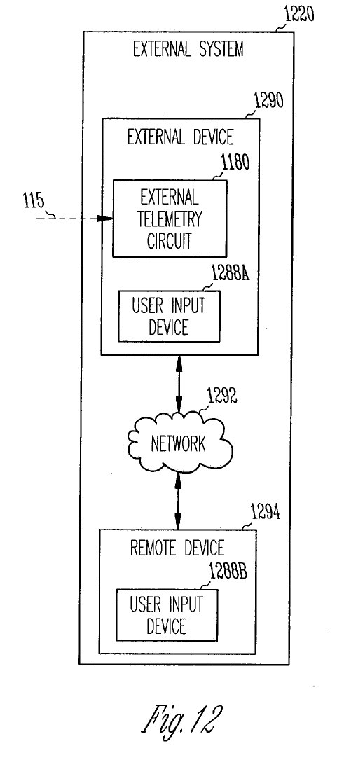

In one embodiment, external system 1120 includes an external device for use by the patient, for example, to initiate and/or time the delivery of the light therapy from implantable medical device 910 as directed by the physician or other caregiver. In another embodiment, external system 1120 includes a patient management system, such as the one discussed below with reference to FIG. 12, that also allows the physician or other caregiver to control the delivery of the light therapy from implantable medical device 910 from a remote location, FIG. 12 is a block diagram illustrating an embodiment of an external system 1220, which represents an embodiment of external system 1120. As illustrated in FIG. 12, external system 1220 is a patient management system including an external device 1290, a telecommunication network 1292, and a remote device 1294. External device 1290 is placed within the vicinity of implantable medical device 910 and includes external telemetry circuit 1180 to communicate with implantable medical device 910 via telemetry link 115. Remote device 1294 is in a remote location and communicates with external device 1290 through network 1292. In the illustrated embodiment, external device 1290 includes a user input device 1288A, and remote device 1294 includes a user input device 1288B. User input device 1288A allows the patient to enter the light emission command. This allows therapy administration at home on a regular basis without the presence of the physician or other professional caregiver. User input device 1288AB allows the patient or other caregiver to enter the light emission command from a remote location, without the need for the patient's presence.

FIGS. 13-15 illustrate various examples of distribution of light emission controller 862 in an implantable medical device and/or an external system communicating with the implantable medical device. In various embodiment, how light emission controller 862 is distributed in an implantable medical device and/or an external system depends on factors including, for example, size of circuitry incorporated into an intravascular stent, energy required for the delivery of the light therapy, and who controls the delivery of the light therapy.

FIG. 13 is a block diagram illustrating an embodiment of an implant control circuit 1370, which represents an embodiment of implant control circuit 970. Implant control circuit 1370 includes light emission controller 862 (including light emission initiator 864 and light emission timer 866). In this embodiment, power supply circuit 1068 includes at least battery 1074.

FIG. 14 is a block diagram illustrating an embodiment of an implant control circuit 1470, which represents an embodiment of implant control circuit 970, and an external control circuit 1482, which represents an embodiment of external control circuit 1182. Light emission controller 862 is distributed in both implant control circuit 970 and external control circuit 1182. Implant control circuit 1470 includes light emission timer 866, and external control circuit 1482 includes light emission initiator 864. In this embodiment, external telemetry circuit 1180 transmits, and implant telemetry circuit 1072 receives, via telemetry link 115 the power transmission signal and the light initiation signal.

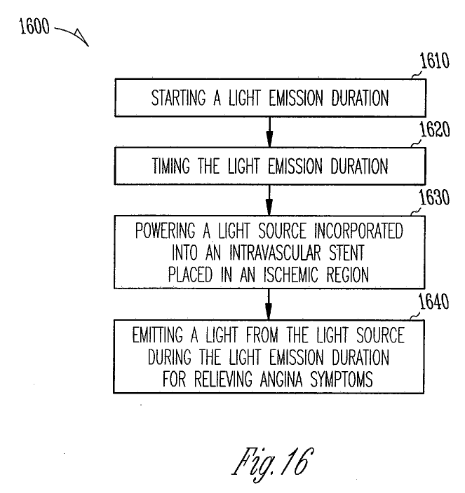

FIG. 15 is a block diagram illustrating an embodiment of an external control circuit 1582, which represents an embodiment of external control circuit 1182. External control circuit 1582 includes light emission controller 862 (including light emission initiator 864 and light emission timer 866). In this embodiment, external telemetry circuit 1180 transmits, and implant telemetry circuit 1072 receives, via telemetry link 115 the power transmission signal and the light emission signal.

FIG. 16 is a flow chart illustrating a method 1600 for relieving angina symptoms using a light therapy. In one embodiment, the method is performed using system 100, including its various embodiments.

At 1610, a light emission duration is started. The light emission duration is a time interval during which a light is emitted to an ischemic region in a patient's body to relieve angina symptoms resulting from ischemia. In one embodiment, the light emission duration is started in response to the light emission command entered by a user, such as the patient, an attendant providing care to the patient, or a physician or other professional caregiver. In another embodiment, the light emission duration is started automatically according to a specified schedule, such as on a periodic basis using a programmed period. In one embodiment, the period is programmable between 1 minute and 72 hours.

At 1620, the light emission duration is timed. In one embodiment, the light emission duration is programmable between 10 seconds and 60 minutes.

At 1630, a light source is powered. The light source is incorporated into an intravascular stent placed in or near the ischemic region in the patient's body. Examples of the light source include one or more LEDs, such as one or more LEDs in die form or one or more PLEDs. In one embodiment, the light source is powered by a power source that is also incorporated into the intravascular stent. In one embodiment, the power source receives a power transmission signal from a device external to the body via a telemetry link capable of power transmission, such as an inductive or ultrasonic couple.

At 1640, a light is emitted from the light source during the light emission duration. The light has characteristics suitable for reliving angina symptoms. In one embodiment, the light has a wavelength between 600 nanometers and 1,000 nanometers, with approximately 800 nanometers being a specific example.

In one embodiment, the light emission duration is started at 1610 and timed at 1620 using circuitry incorporated into the intravascular stent. In another embodiment, the light emission duration is started at 1610 using an external system communicatively coupled to circuitry incorporated into the intravascular stent, and timed at 1620 using circuitry incorporated into the intravascular stent. In another embodiment, the light emission duration is started at 1610 and timed at 1620 using the external system communicatively coupled to the circuitry incorporated into the intravascular stent.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A system for reliving angina symptoms in a body, the system comprising:
an implantable medical device including
an intravascular stent; and
a light emitting circuit incorporated into the intravascular stent, the light emitting circuit including:
a light source adapted to emit a light having characteristics suitable for reliving the angina symptoms;
a power supply circuit adapted to produce a power supply signal suitable for powering the light source; and
an implant control circuit coupled to the light source and the power source, the implant control circuit adapted to control the emission of the light from the light source during a light emission duration.

2. The system of claim 1, wherein the light emitting circuit is constructed on a tubular substrate coupled to an end portion of the intravascular stent.

3. The system of claim 1, wherein the light emitting circuit is constructed on one or more substrate portions etched on portions of the intravascular stent.

4. The system of claim 1, wherein the light source comprises one or more light emitting diodes (LEDs).

5. The system of claim 3, wherein the one or more LEDs comprise one or more polymeric light emitting diodes (PLEDs).

6. The system of claim 4, wherein the one or more LEDs comprise one or more LEDs each configured to emit a light having a wavelength between 600 nanometers and 1,000 nanometers.

7. The system of claim 1, wherein the implant control circuit comprises:
a light emission initiator adapted to start the light emission duration; and
a light emission timer adapted to time the light emission duration.

8. The system of claim 7, wherein the light emission initiator is adapted to receive a light emission command and start the light emission duration in response to the light emission command.

9. The system of claim 7, wherein the light emission initiator is adapted to start the light emission command according to a specified schedule.

10. The system of claim 7, comprising an external system communicatively coupled to the implantable medical device, the external system including:
an external telemetry circuit adapted to transmit one or more of a data transmission signal and the power transmission signal to the implantable medical device; and
an external control circuit coupled to the external telemetry circuit,
and wherein the implantable medical device comprises an implantable telemetry circuit coupled to the implant control circuit, the implantable telemetry circuit adapted to receive the one or more of the data transmission signal and the power transmission signal.

11. The system of claim 10, wherein the external system comprises a power transmitter coupled to the external telemetry circuit and adapted to transmit the power transmission signal to the implantable medical device, and wherein the power supply circuit of the implantable medical device comprises:
a power receiver adapted to receive the power transmission signal; and
a power converter adapted to produce the power supply signal using the power transmission signal.

12. The system of claim 11, wherein the light emission initiator is adapted to receive a light emission command and produce the light initiation signal in response to the light emission command, and the external system comprises a user input device to receive the light emission command.

13. The system of claim 11, wherein:
the implant control circuit comprises the light emission timer;
the implant telemetry circuit is configured to receive the power transmission signal and the light initiation signal;
the external control circuit comprises the light emission initiator; and
the external telemetry circuit is configured to transmit the power transmission signal and the light initiation signal.

14. The system of claim 11, wherein:
the external control circuit comprises the light emission initiator and the light emission timer;
the implant telemetry circuit is configured to receive the power transmission signal and the light emission signal; and
the external telemetry circuit is configured to transmit the power transmission signal and the light emission signal.

15. A method for relieving angina symptoms in a body having an ischemic region, the method comprising:
starting a light emission duration;
timing the light emission duration;
powering a light source using a power source, the light source and the power source both incorporated into an intravascular stent configured to be placed in the ischemic region; and
emitting a light from the light source during the light emission duration, the light having characteristics suitable for reliving the angina symptoms.

16. The method of claim 15, wherein starting the light emission duration comprises starting the light emission duration in response to a light emission command entered by a user.

17. The method of claim 15, wherein starting the light emission duration comprises starting the light emission duration on a periodic basis using a programmed period.

18. The method of claim 17, comprising programming the period to a value between 1 minute and 72 hours.

19. The method of claim 15, comprising programming the light emission duration to a value between 10 seconds and 60 minutes.

20. The method of claim 15, wherein emitting the light comprises emitting a light having a wavelength between 600 nanometers and 1,000 nanometers.

21. The method of claim 20, wherein emitting the light from the light source comprises emitting the light from one or more light emitting diodes (LEDs) incorporated into the intravascular stent.

22. The method of claim 21, wherein emitting the light from the light source comprises emitting the light from one or more polymeric light emitting diodes (PLEDs).

23. The method of claim 15, comprising receiving power from a device external to the body via a telemetry link capable of power transmission.

24. The method of claim 15, wherein timing the light emission duration comprises timing the light emission duration using circuitry incorporated into the intravascular stent.

25. The method of claim 24, wherein starting the light emission duration comprises starting the light emission duration using circuitry incorporated into the intravascular stent.

26. The method of claim 24, wherein starting the light emission duration comprises starting the light emission duration using an external system communicatively coupled to the circuitry incorporated into the intravascular stent.

27. The method of claim 15, wherein timing the light emission duration comprises timing the light emission duration using an external system communicatively coupled to the circuitry incorporated into the intravascular stent, and starting the light emission duration comprises starting the light emission duration using the external system.
